(19) United States
(12) Patent Application Publication
(10) Pub. No.: US 2026/0199558 A1
(43) Pub. Date: Jul. 16, 2026

(54) INJECTABLE COMPOSITIONS FOR TREATING JOINT CONDITIONS

(71) Applicant: Ohio State Innovation Foundation, Columbus, OH (US)

(72) Inventors: Katelyn Elizabeth REILLY, Columbus, OH (US); Devina WALTER, Columbus, OH (US)

(21) Appl. No.: 19/138,457

(22) PCT Filed: Dec. 14, 2023
(86) PCT No.: PCT/US23/84018

§ 371 Date: Jun. 12, 2025

Related U.S. Application Data
(60) Provisional application No. 63/432,726, filed on Dec. 15, 2022.

(51) Int. Cl. A61L 27/52; A61L 27/16; A61L 27/20; A61L 27/22; A61L 27/24; C07K 16/22

(52) U.S. Cl. CPC A61L 27/52; A61L 27/16; A61L 27/20; A61L 27/225; A61L 27/24; C07K 16/22; A61L 2300/222; A61L 2300/256; A61L 2300/41; A61L 2300/45; A61L 2400/06; A61L 2400/12; A61L 2430/24

(57) ABSTRACT

Methods of treating joint conditions are described. In some implementation, the method includes injecting into an affected joint of the subject a therapeutically effective amount of a therapeutic composition which may include a hydrogel: and a second agent dispersed within the hydrogel selected from one or more antioxidants, one or more reactive oxygen species (ROS) scavengers, or combinations thereof. Injectable compositions useful in treating joint conditions are also provided.

### CROSS-REFERENCE TO RELATED APPLICATIONS

This application claims the benefit of priority to U.S. Provisional Application No. 63/432,726 filed Dec. 15, 2022, the disclosure of which is incorporated herein by reference in its entirety.

### TECHNICAL FIELD

This disclosure relates to compositions and methods for treating medical disorders and, more particularly, to compositions and methods for treating joint conditions.

### BACKGROUND

Joint conditions, also known as arthropathies, are common medical disorders that may arise as a natural result of normal degeneration during the aging process or secondary to other disease states, such as inflammatory or immune disorders or physical injury. While intraarticular injections are a common therapy used for such conditions, those currently available often only offer limited short-term efficacy in terms of symptom relief and do little to counteract the physiological processes leading to the joint condition. For example, corticosteroid injections may provide some pain relief in degenerative disc disease or osteoarthritis, but typically only in the short term, and injections of hyaluronic acid for knee or ankle arthritis have shown limited improvement with increased risk for further pain. There is a clear need for new methods and compositions which may be used in the treatment of joint conditions.

### SUMMARY

The present disclosure provides methods for treating joint conditions, along with injectable compositions useful in treating joint conditions. The present disclosure also provides compositions for use in treating joint conditions, use of said compositions in treating joint conditions, and use of said compositions in the manufacture of medicaments for treating joint conditions.

In one general aspect, methods of treating a joint condition in a subject in need thereof are provided. In some implementations, the method may include injecting into an affected joint of the subject a therapeutically effective amount of a therapeutic composition comprising: a hydrogel; and a second agent dispersed within the hydrogel selected from one or more antioxidants, one or more reactive oxygen species (ROS) scavengers, or combinations thereof. In other implementations, the method may include injecting into an affected joint of the subject a therapeutically effective amount of a therapeutic composition comprising: one or more ROS scavengers and one or more therapeutic agents.

In another general aspect, injectable compositions are provided. In some implementations, the injectable compositions may include: a hydrogel including one or more residues selected from poly(ethylene glycol)diacrylate (PEDGA), poly(ethylene glycol)methacrylate (PEGMA), 2-hydroxyethylmethacrylate (HEMA), or combinations thereof; and a reactive oxygen species (ROS) scavenger comprising a plurality of particles. In other implementations, the injectable compositions may include: a hydrogel including a mixture or copolymer of a polyalkylene glycol and a second component selected from hyaluronate, fibrin, alginate, or collagen; and a second agent dispersed within the hydrogel selected from one or more antioxidants, one or more reactive oxygen species (ROS) scavengers, or combinations thereof. Methods of using the described injectable compositions for treating joint conditions are also provided.

The details of one or more aspects of the disclosure are set forth in the description below. Other features, objects, and advantages of the disclosure will be apparent from the description, the figures, and the claims.

### DESCRIPTION OF DRAWINGS

FIGS. 1A and 1B depict robust gene expression of vascular endothelial growth factor A (VEGFA) in both human cartilage endplate (CEP) cells and annulus fibrosus (AF) cells. FIG. 1A provides the average CT values. FIG. 1B provides the values normalized to the housekeeping gene 18s with the delta CT values.

FIGS. 2A and 2B provide quantified dose effects (FIG. 2A) and representative images (FIG. 2B) of PDA nanoparticle on human cartilage endplate (CEP) cell viability. High viability (≥80%) was observed for most doses other than 200 μg/mL (approximately 60%).

FIGS. 3A and 3B provide quantified dose effects (FIG. 3A) and representative images (FIG. 3B) of PDA nanoparticle on human annulus fibrosus (AF) cell viability. High viability (≥80%) was observed for most doses.

FIGS. 4A and 4B provide quantified dose effects (FIG. 4A) and representative images (FIG. 4B) of free bevacizumab on human cartilage endplate (CEP) cell viability. Cell viability was >80% up to 0.5 mg/mL but then dropped to 30% with 1 mg/mL dosing.

FIGS. 5A and 5B provide quantified dose effects (FIG. 5A) and representative images (FIG. 5B) of free bevacizumab on human annulus fibrosus (AF) cell viability. High viability (75%) was maintained at 1 mg/mL but dropped to approximately 30% at higher doses.

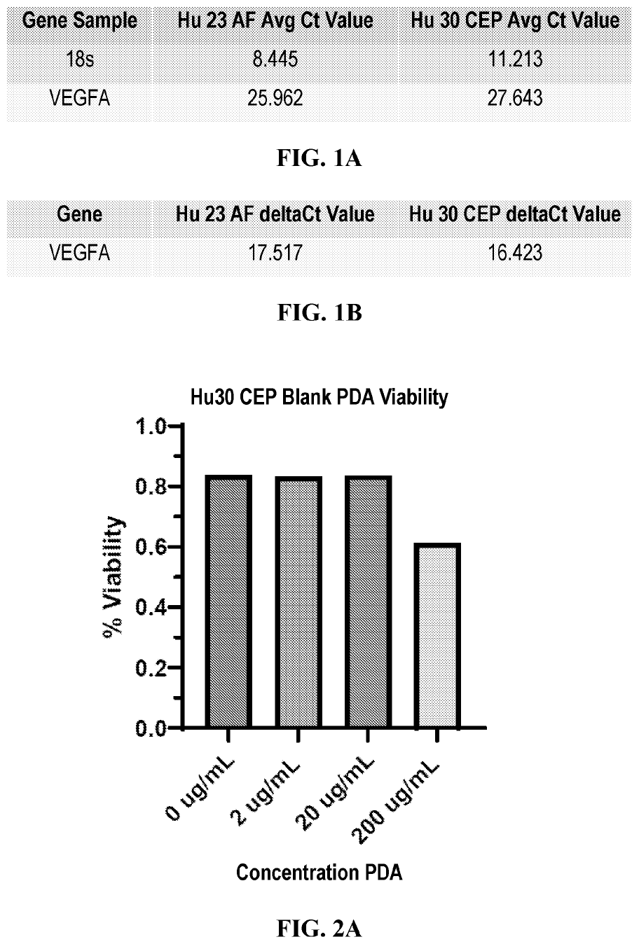

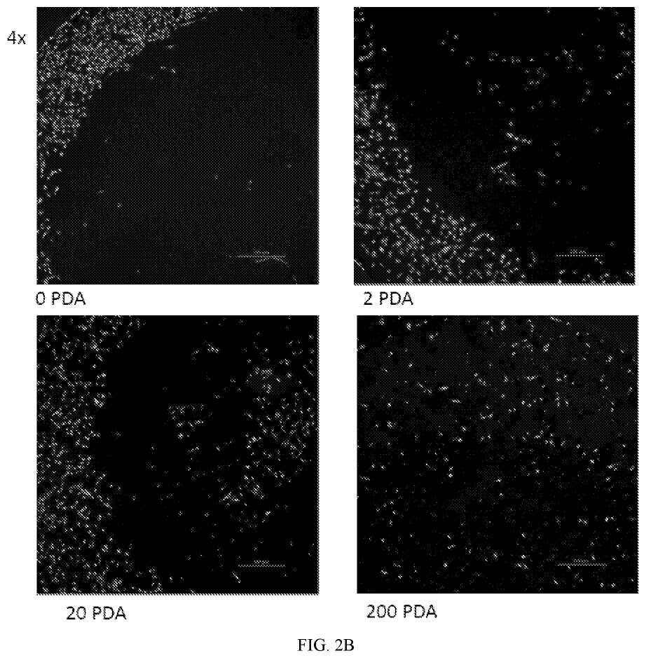

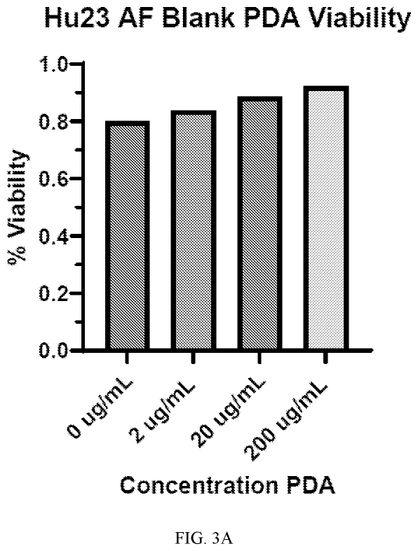

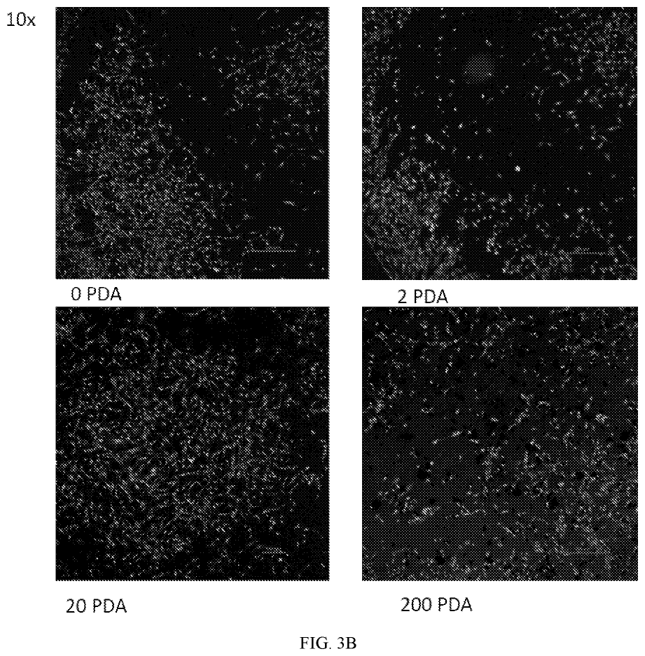

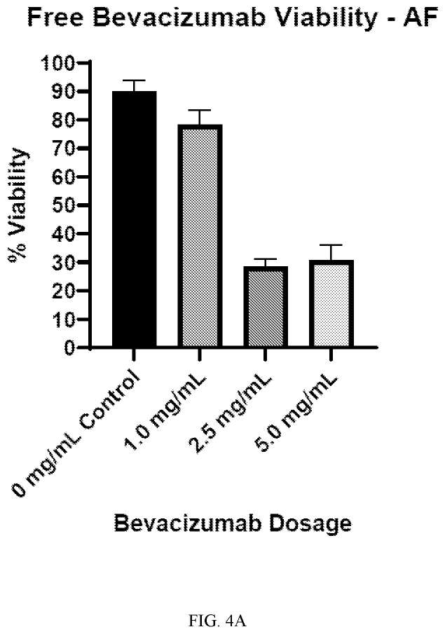

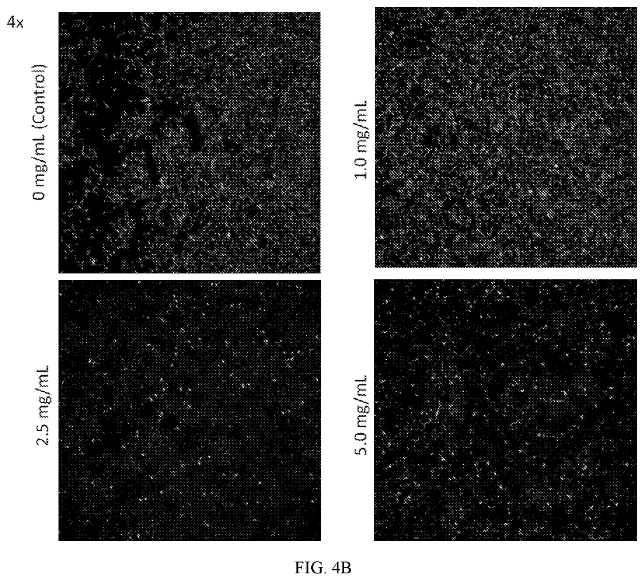

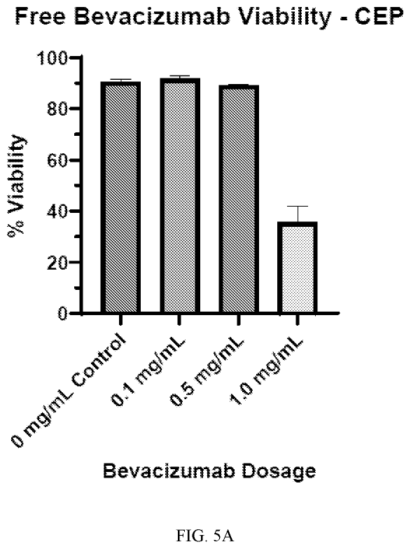

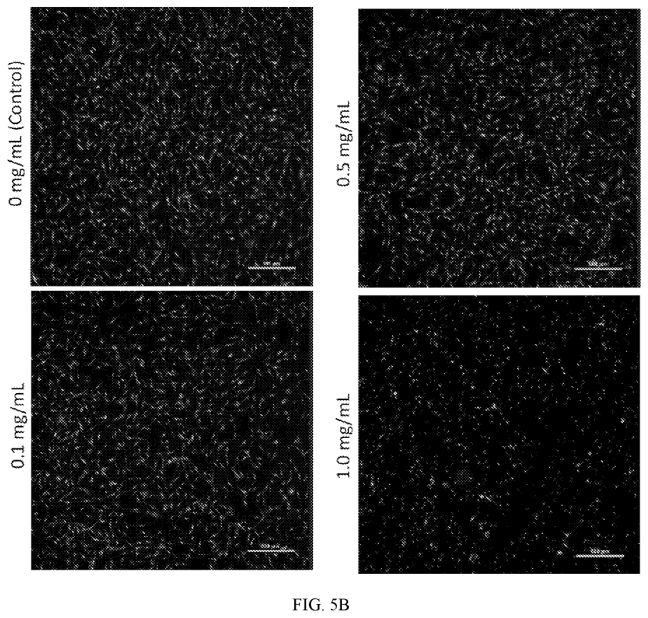

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present disclosure is not entitled to antedate such publication. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” an, and “the” include plural referents unless the context dictates otherwise. Thus, for example, reference to “a composition,” “a joint,” or “a condition” includes, but is not limited to, two or more such compositions, joints, or conditions, and the like.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single-dose compositions can contain such amounts or submultiples thereof to make up the daily dose. The individual physician can adjust the dosage in the event of any contraindications. It is generally preferred that a maximum dose of the pharmacological agents of the disclosure (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition such as a joint condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

In a first general aspect, a method of treating a joint condition in a subject in need thereof is provided. In some implementations, the method may include injecting into an affected joint of the subject a therapeutically effective amount of a therapeutic composition which may include: a hydrogel; and a second agent dispersed within the hydrogel selected from one or more antioxidants, one or more reactive oxygen species (ROS) scavengers, or combinations thereof. In other implementations, the method may include injecting into an affected joint of the subject a therapeutically effective amount of a therapeutic composition comprising: one or more reactive oxygen species (ROS) scavengers and one or more therapeutic agents.

In some aspects, the second agent can have a concentration greater than about 1% by weight within the therapeutic composition, for example, greater than about 1%, about 2%, about 3%, about 4%, about 5%, about 6%, about 7%, about 8%, about 9%, about 10%, about 12%, about 14%, about 16%, about 18%, about 20%, about 25%, or about 30% by weight within the therapeutic composition. In some aspects, the second agent can have a concentration from about 1% to about 30% by weight within the therapeutic composition, for example, from about 1% to about 5%, from about 1% to about 10%, from about 1% to about 15%, from about 1% to about 20%, from about 1% to about 25%, from about 1% to about 30%, from about 5% to about 10%, from about 5% to about 15%, from about 5% to about 20%, from about 5% to about 25%, from about 5% to about 30%, from about 10% to about 15%, from about 10% to about 20%, from about 10% to about 25%, from about 10% to about 30%, from about 15% to about 20%, from about 15% to about 25%, from about 15% to about 30%, from about 20% to about 25%, from about 20% to about 30%, or from about 25% to about 30% by weight within the therapeutic composition.

In some aspects, the second agent may include one or more antioxidants. Any suitable antioxidant can be used as a therapeutic agent in the disclosed compositions. As used herein, it should be understood that the use of the term “antioxidant” is inclusive of free-radical scavengers and can be used interchangeably with “free-radical scavenger.” The term “free-radical scavenger,” as used herein, refers to a substance, such as an antioxidant, that helps protect cells from the damage caused by free radicals.

In some aspects, the antioxidant is present in an amount sufficient to produce a therapeutic effect without showing any significant toxicity to the tissues of the joint.

In some aspects, the antioxidant used can include vitamin A; vitamin C (ascorbic acid); N-acetylcysteine; glutathione; a zinc compound; a copper compound; vitamin E and derivatives thereof, including, but not limited to, alpha, beta, gamma, and delta tocopherol and/or alpha, beta, gamma, and delta tocotrienols, and derivatives thereof; selenous acid; sodium selenite; a saturated and unsaturated fatty acid, including, but not limited to, 6-O-lauroyl ascorbate, 6-O-myristoyl ascorbate, 6-O-oleoyl ascorbate, 6-O-palmitoyl ascorbate, 6-O-linoleoyl ascorbate, 6-O-stearoyl ascorbate; 1-carnitine and derivatives such as 1-carnitine acetate; retinal; tretinoin; timolol; lutein; thyroxine; pyrroloquinolone; probucol; captopril; uric acid; erithorbic acid and its salts; α-lipoic acid; hydralazine; gallic acid; lycopene; astaxanthin; zeaxanthin; ferulic acid; quercetin; eugenol; isoeugenol; melatonin; resveratrol; mannitol; trolox; methylethylpiridinol; taufon; a thiol antioxidant; beta carotene; and combinations of one or more of the foregoing.

In a further aspect, the antioxidant used can include vitamin E; vitamin C (ascorbic acid); lutein; zeaxanthin; a zinc compound; a copper compound; beta carotene; one or more omega-3 fatty acid, e.g., DHA or EPA; or combinations thereof.

In some aspects, the antioxidant used can include alpha lipoic acid, riboflavin, taurine, uric acid, tyrosine, transferring, selenium, zinc, superoxide dismutase, glutathione peroxidase, catalase, pigment epithelium-derived factor (PEDF), or combinations thereof.

In a further aspect, a thiol antioxidant can be selected from glutathione (GSH), oxidation-type glutathione or oxidized glutathione (GSSG), N-acetylcysteine, thioctic acid, 2-oxo-thiazolidine-4-carboxylic acid, cysteine, glutamylcysteine, ethanethiol, 1,4-butanethiol, 2-mercaptoethylether, pentaerythretoltetrathiopropionate and acetate, polyethyleneglycolimercaptoacetate and methylthioglycolate, allyl mercaptan, 2-mercaptoethanol, 3-mercaptopropanol, 4-mercaptobutanol, 1-thioglycerol, thioerythritol, 2,3-dimercaptopropanol, pentaerythretolmono (di; tri)thiopropionate or acetate, thioglycolic acid, thioacetic acid, 3-mercaptopropionic acid, thiolactic acid, thiomalic acid, thiosuccinic acid, thiosalicylic acid, thiobenzoic acid and their respective water soluble salts, furfuryl mercaptan, 2-mercaptobenzimidazole, 2-mercaptobenzoxazole, 2-mercapto-3-pyridinol, dimethylaminopropanethiol, 2-mercaptoethylamine, 2-n-butylaminoethanethiol; derivatives of the foregoing; and mixtures of the foregoing or in combination with another disclosed antioxidant thereof.

In a further aspect, a thiol antioxidant can be selected from N-acetylcysteine, thioctic acid, 2-oxo-thiazolidine-4-carboxylic acid, cysteine, glutamylcysteine, and mixtures thereof.

In a further aspect, a thiol antioxidant can be selected from GSH, pharmaceutically acceptable salts of GSH, GSSG, pharmaceutically acceptable salts of GSSG, precursors thereof, and mixtures thereof. In a still further aspect, a thiol antioxidant can be selected from GSH, GSSG, pharmaceutically acceptable salts thereof, and mixtures thereof. In a yet further aspect, a thiol antioxidant can be selected from GSH, GSSG, and mixtures thereof. In an even further aspect, a thiol antioxidant comprises GSH.

In a further aspect, pharmaceutically acceptable anions included in the pharmaceutically acceptable salts of an antioxidant include chloride, bromide, iodide, sulfate, bisulfate, phosphate, acid phosphate, nitrate, acetate, maleate, fumarate, oxalate, lactate, tartrate, citrate, gluconate, saccharate, p-toluene sulfonate and the like. Pharmaceutically acceptable derivatives useful as an antioxidant include esters, acids, and the like.

In other aspects, the antioxidant present in a disclosed composition can be one or more of an agent selected from ascorbic acid, Na ascorbate, K ascorbate, Ca ascorbate, Mg ascorbate, Zn ascorbate; 6-O-esters of ascorbic acid with C2 to C20 straight, branched, saturated and unsaturated fatty acids: 6-O-lauroyl ascorbate, 6-O-myristoyl ascorbate, 6-O-oleoyl ascorbate, 6-O-palmitoyl ascorbate, 6-O-linoleoyl ascorbate, 6-O-stearoyl ascorbate; 6-O-ester of ascorbic acid with d, or dl-α-tocopheryl hemisuccinate; 6-O-esters of ascorbic acid with reduced glutathione and d, or dl-α-tocopherols; reduced glutathione and glutathione ester of reduced glutathione with d or dl-α-tocopherol; d and dl-tocopherol (α, β, γ, δ isomers) and the acetate, hemisuccinate, nicotinate, and succinate-PEG ester (TPGS) derivatives of the foregoing tocopherol isomers; superoxide dismutase; β-carotene; melatonin; trans resveratrol; trolox; coenzyme Q; catalase; various peroxidases; cysteine, ester of cysteine with ethanol, HCl salt of the ester of cysteine with ethanol, the salt of ascorbic acid with the ester of cysteine with ethanol, the d or dl-α-tocopherol-hemisuccinate salt of the ester of cysteine with ethanol, the ester of cysteine with d, or dl-α-tocopherol, N-acetylcysteine, Na, K, Ca, Mg, Zn salts of N-acetylcysteine, ester of N-acetyl cysteine with ethanol or d, or dl-α-tocopherol; 1-carnitine; 1-carnitine acetate; retinal; tretinoin; timolol; lutein; thyroxine; pyrroloquinolone; probucol; captopril; desferal Mn+3; uric acid; erithorbic acid and its salts; α-lipoic acid; lycopene; astaxanthin; zeaxanthin; ferulic acid; quercetin; eugenol and isoeugenol; prostaglandins; latanoprost, bimatoprost, travoprost; (−)-epicatechin; (−)-epigallocatechin gallate; butylated hydroxytoluene; butylated hydroxyanisole; rutinal; fisetin; sulfite and bisulfite salts (Na, K, Ca, Mg). In some aspects, the antioxidant may include L-ascorbic acid, ascorbic acid 6-palmitate, or combinations thereof.

In some aspects, the antioxidant present in the disclosed compositions may include one or more of the ascorbic acid derivatives described in Macan, A. et al. “Therapeutic Perspective of Vitamin C and Its Derivatives” Antioxidants 2019, 8, 247, doi:10.3390/antiox8080247, incorporated herein by reference in its entirety for all purposes.

Representative examples of antioxidants that may be used include, but are not limited to: N-acetylcysteine; a glutathione; N-selenous acid; sodium selenite; L-carnitine; β-carotene; vitamin E; ascorbic acid or a derivative thereof; lutein; zeaxanthin; an omega-3 fatty acid; alpha lipoic acid; riboflavin; taurine; uric acid; tyrosine; transferrin; selenium; zinc; superoxide dismutase; glutathione peroxidase; catalase; pigment epithelium-derived factor (PEDF); or combinations thereof.

In some aspects, the one or more antioxidants may include ascorbic acid or a derivative thereof. In some aspects, the one or more antioxidants may include a glutathione. In some aspects, the one or more antioxidants may include a combination of ascorbic acid or a derivative thereof and a glutathione.

In some aspects, the second agent may include one or more reactive oxygen species (ROS) scavengers. In some aspects, the one or more reactive oxygen species (ROS) scavengers may include a plurality of particles. Representative examples of particles that can be used include, but are not limited to, polydopamine (PDA) nanoparticles, cerium oxide particles, polyphenylene sulfide (PPS) particles, phenolic nanoparticles, or combinations thereof. In some aspects, the second may include a combination of one or more antioxidants, as described herein, and one or more ROS scavengers, such as a plurality of particles. In some aspects, the plurality of particles further includes (e.g., encapsulates) the one or more antioxidants.

In some particular aspects, the plurality of particles may include polydopamine (PDA) particles. Polydopamine is formed by the oxidation of dopamine. It is biomimetic of the proteins on the extremity of mussel byssus, which are extremely rich in L-DOPA and L-Lysine residues. These amino acid residues, containing catechol and amino functional groups, allow for strong adhesion of the mussel to all kinds of substrates in the wet and slightly basic environment of seawater. Because of this, polydopamine has traditionally found extensive use in adhesive coatings. Methods for forming polydopamine nanoparticles are known in the art. Typically, polydopamine nanoparticles are formed from alkaline aqueous solutions (for example, in the presence of Tris buffer at pH=8.5) in the presence of an oxidant. Oxygen dissolved in the aqueous solution is typically used as the oxidant, but other oxidants may be used, for example, ammonium peroxodisulfate or sodium periodate. In some aspects, the polydopamine nanoparticles, as used in the present disclosure, are essentially spherical, spheroid, ellipsoid, or combinations thereof.

In some aspects, the polydopamine particles as used in the present disclosure have an average particle size ranging from about 10 nm to about 1000 nm, for example from about 100 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 900 nm to about 1000 nm, from about 10 nm to about 900 nm, from about 100 nm to about 900 nm, from about 200 nm to about 900 nm, from about 300 nm to about 900 nm, from about 400 nm to about 900 nm, from about 500 nm to about 900 nm, from about 600 nm to about 900 nm, from about 700 nm to about 900 nm, from about 800 nm to about 900 nm, from about 10 nm to about 800 nm, from about 100 nm to about 800 nm, from about 200 nm to about 800 nm, from about 300 nm to about 800 nm, from about 400 nm to about 800 nm, from about 500 nm to about 800 nm, from about 600 nm to about 800 nm, from about 700 nm to about 800 nm, from about 10 nm to about 700 nm, from about 100 nm to about 700 nm, from about 200 nm to about 700 nm, from about 300 nm to about 700 nm, from about 400 nm to about 700 nm, from about 500 nm to about 700 nm, from about 600 nm to about 700 nm, from about 10 nm to about 600 nm, from about 100 nm to about 600 nm, from about 200 nm to about 600 nm, from about 300 nm to about 600 nm, from about 400 nm to about 600 nm, from about 500 nm to about 600 nm, from about 10 nm to about 500 nm, from about 100 nm to about 500 nm, from about 200 nm to about 500 nm, from about 300 nm to about 500 nm, from about 400 nm to about 500 nm, from about 10 nm to about 400 nm, from about 100 nm to about 400 nm, from about 200 nm to about 400 nm, from about 300 nm to about 400 nm, from about 10 nm to about 300 nm, from about 100 nm to about 300 nm, from about 200 nm to about 300 nm, from about 10 nm to about 200 nm, from about 100 nm to about 200 nm, or from about 10 nm to about 100 nm.

In some aspects, the population of polydopamine nanoparticles has an average particle size of about 10 nm, about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm.

In some aspects, the polydopamine particles, as described herein, may include a coating. The coating can be disposed on the surface of the particle, for example, by bonding, adsorption, or complexation. The coating can also be intermingled or dispersed within the particle as well as disposed on the surface of the particle.

In some aspects, the coating may include a polymer, i.e., the polydopamine particles as described herein may be coated with a polymer. In some aspects, the polymer may include an alginate. In some aspects, the polymer may include polyethylene glycol, polyvinyl alcohol, polycaprolactone, polylactic acid, polyglycolic acid, poly(lactic-co-glycolic acid), or similar substances. In some aspects, the coating may also include a non-ionic surfactant such as those composed of polyalkylene oxide, e.g., polyethylene glycol or polypropylene glycol, and can include a copolymer of more than one alkylene oxide. In some aspects, the coating can include a polyoxyethylene-polyoxypropylene copolymer, i.e., a poloxamer such as poloxamer 188, 237, 338, and 407.

In some aspects, the plurality of particles may include one or more therapeutic agents. Suitable therapeutic agents include any which may be useful in the treatment of a joint condition in a subject in need thereof.

Representative therapeutic agents which may be used include, but are not limited to:
anti-interleukin antibodies (for example, clazakizumab, elsilimomab, levelimab, olokizumab, sarilumab, secukinumab, siltuximab, sirukumab, tocilizumab, or ustekinumab);
tyrosine kinase inhibitors (for example, avapritinib, capmatinib, pemigatinib, ripretinib, selpercatinib, selumetinib, tucatinib, entrectinib, erdafitinib, fedratinib, pexidartinib, upadacitinib, zanubrutinib, baricitinib, binimetinib, dacomitinib, fostamatinib, gilteritinib, larotrectinib, lorlatinib, acalabrutinib, brigatinib, midostaurin, neratinib, alectinib, cobimetinib, Lenvatinib, osimertinib, ceritinib, nintedanib, afatinib, ibrutinib, trametinib, axitinib, bosutinib, cabozantinib, ponatinib, regorafenib, tofacitinib, crizotinib, ruxolitinib, vandetanib, pazopanib, lapatinib, nilotinib, dasatinib, sunitinib, sorafenib, erlotinib, gefitinib, or imatinib);
complement inhibitors (for example, avacopan, eculizumab, pegcetacoplan, ravulizumab, or sutimlimab);
MAPK inhibitors;
JAK inhibitors;
interferons (for example, interferon alpha, interferon alpha A, interferon beta, interferon gamma, or interferon-1 alpha);
anti-VEGF agents (for example, aflibercept, axitinib, bevacizumab, cabozantinib, lapatinib, lenvatinib, pazopanib, ponatinib, ramucirumab, ranibizumab, regorafenib, sorafenib, sunitinib, tivozanib, or vandetanib);
anti-NGF agents (for example, fasinumab, frunevetmab, fulranumab, GBR-900, GZ-389988, LEVI-04, NRD135S-E1, ONO-4494, ranevetmab, tanezumab, or VM-902A);
senolytics; or combinations thereof.

In some aspects, the therapeutic agent may include a nonsteroidal anti-inflammatory drug, a steroid, a disease-modifying antirheumatic drug, or combinations thereof.

Representative examples of nonsteroidal anti-inflammatory drugs which can be used include, but are not limited to, aspirin, diflunisal, salsalate, ibuprofen, flurobiprofen, dexibuprofen, oxaprozin, naproxen, loxoprofen, fenoprofen, pelubiprofen, ketoprofen, zaltoprofen, dexketoprofen, indomethacin, etodolac, aceclofenac, tolmetin, ketorolac, bromfenac, sulindac, diclofenac, nabumetone, piroxicam, droxicam, phenylbutazone, meloxicam, lornoxicam, tenoxicam, isoxicam, mefenamic acid, flufenamic acid, meclofenamic acid, tolfenamic acid, celocoxib, parecoxib, firocoxib, rofecoxib, lumiracoxib, valdecoxib, etoricoxib, nimesulide, clonixin, or licofelone.

Representative examples of steroids that can be used include, but are not limited to, beclomethasone, betamethasone, budesonide, cortisone, dexamethasone, hydrocortisone, methylprednisolone, prednisolone, prednisone, and triamcinolone.

Representative examples of disease-modifying antirheumatic drugs which can be used include, but are not limited to, abatacept, adalimumab, anakinra, apremilast, azathioprine, baricitinib, certolizumab pegol, chloroquine, ciclosporin, D-penicillamine, etanercept, filgotinib, golimumab, sodium aurothiomalate, auranofin, hydroxychloroquine, infliximab, leflunomide, methotrexate, minocycline, rituximab, sarilumab, secukinumab, sulfasalazine, tocilizumab, tofacitinib, and ustekinumab.

In various aspects, the hydrogel may include a polymer composition, for example, a homopolymer, a copolymer, or combinations thereof. In some instances, the hydrogel may include a copolymer.

In some aspects, the hydrogel may include one or more hydrophilic polymers. A hydrophilic polymer may be defined as a polymer having at least 0.1 wt % solubility in water, for example, having at least 0.5 wt % solubility. In some aspects, the hydrophilic polymer has a solubility of at least 1 mg/mL in water.

In some aspects, the polymer composition may include one or more vinyl alcohol residues. In some aspects, the polymer composition may include one or more acrylamide residues. In some aspects, the polymer composition may include one or more residues selected from a polyethylene glycol derivative or a functionalized polyethylene glycol. In some aspects, the polymer composition may include one or more acrylate residues or one or more methacrylate residues. In some aspects, the polymer composition may include one or more residues selected from acrylamide, N-ornithine acrylamide, N-(2-hydroxypropyl)acrylamide, hydroxyethylacrylate, hydroxyethylmethacrylate, polyethyleneglycol acrylates, polyethylene glycol methacrylates, N-vinylpyrrolidone, N-phenylacrylamide, dimethylaminopropyl methacrylamide, acrylic acid, benzylmethacrylamide, methylthioethylacrylamide, caprolactone, lactic acid, glycolic acid, or combinations thereof.

In some aspects, the polymer composition can be a copolymer which may include residues derived from HEMA, PEGDA, and/or PEGMA as described herein.

In some aspects, the disclosed hydrogels comprise a polymer prepared utilizing one or more of: 2-hydroxyethyl methacrylate (HEMA) and/or poly(ethylene glycol) methacrylate (PEGMA).

In other aspects, the disclosed hydrogels may include a copolymer prepared utilizing one or more of the following monomers: 2-hydroxyethyl methacrylate (HEMA) and/or poly(ethylene glycol) methacrylate (PEGMA). In a further aspect, the copolymer can be prepared by utilizing a cross-linking agent, e.g., poly(ethylene glycol) diacrylate (PEGDA) crosslinker.

In a further aspect, disclosed hydrogels can be prepared by free radical polymerization of HEMA, PEGMA, and PEGDA. Briefly, HEMA:PEGMA copolymer hydrogels can be polymerized in water and crosslinked with PEGDA. Ammonium persulfate and N,N,N′,N′-Tetramethylethylenediamine are used to initiate and catalyze the reaction. In some instances, the disclosed methods of making a disclosed hydrogel comprise steps as described in the Examples herein, as described in published protocols (A. Zellander, et al., PLoS one. 2014, 9, e96709), in modifications of published protocols, including those described herein, and method optimization thereof as in keeping with the spirit and scope of the present disclosure.

In various aspects, the disclosed hydrogel can be a polymer having one or more PEGDA residues. A disclosed hydrogel including a polymer having one or more PEGDA residues can be formed using the described methods in which polymerization is carried out using PEGDA monomers at a concentration of greater than or equal to about 1 wt % and less than or equal to about 50 wt %, for example, from about 1 wt % to about 40 wt %, from about 1 wt % to about 30 wt %, from about 1 wt % to about 20 wt %, from about 1 wt % to about 10 wt %, from about 1 wt % to about 5 wt %, from about 5 wt % to about 50 wt %, from about 5 wt % to about 40 wt %, from about 5 wt % to about 30 wt %, from about 5 wt % to about 20 wt %, from about 5 wt % to about 10 wt %, from about 10 wt % to about 50 wt %, from about 10 wt % to about 40 wt %, from about 10 wt % to about 30 wt %, from about 10 wt % to about 20 wt %, from about 20 wt % to about 50 wt %, from about 20 wt % to about 40 wt %, from about 20 wt % to about 30 wt %, from about 30 wt % to about 50 wt %, from about 30 wt % to about 40 wt %, or from about 40 wt % to about 50 wt %.

In some aspects, each of the one or more PEDGA residues may independently have a molecular weight of from about 100 to about 10000. In some aspects, each of the one or more PEGDA residues may have a molecular weight of from about 100 to about 1000. In some aspects, each of the one or more PEGDA residues have a molecular weight of from about 100 to about 1000, from about 200 to about 1000, from about 300 to about 1000, from about 400 to about 1000, from about 500 to about 1000, from about 600 to about 1000, from about 700 to about 1000, from about 800 to about 1000, from about 900 to about 1000, from about 100 to about 900, from about 200 to about 900, from about 300 to about 900, from about 400 to about 900, from about 500 to about 900, from about 600 to about 900, from about 700 to about 900, from about 800 to about 900, from about 100 to about 800, from about 200 to about 800, from about 300 to about 800, from about 400 to about 800, from about 500 to about 800, from about 600 to about 800, from about 700 to about 800, from about 100 to about 700, from about 200 to about 700, from about 300 to about 700, from about 400 to about 700, from about 500 to about 700, from about 600 to about 700, from about 100 to about 600, from about 200 to about 600, from about 300 to about 600, from about 400 to about 600, from about 500 to about 600, from about 100 to about 500, from about 200 to about 500, from about 300 to about 500, from about 400 to about 500, from about 100 to about 400, from about 200 to about 400, from about 300 to about 400, from about 100 to 300, from about 200 to 300, or from about 100 to 200.

In various aspects, the disclosed hydrogel can be a polymer having one or more PEGMA residues. A disclosed hydrogel including a polymer having one or more PEGMA residues can be formed using the described methods in which polymerization is carried out using PEGMA monomers at a concentration of greater than or equal to about 1 wt % and less than or equal to about 50 wt %, for example, from about 1 wt % to about 40 wt %, from about 1 wt % to about 30 wt %, from about 1 wt % to about 20 wt %, from about 1 wt % to about 10 wt %, from about 1 wt % to about 5 wt %, from about 5 wt % to about 50 wt %, from about 5 wt % to about 40 wt %, from about 5 wt % to about 30 wt %, from about 5 wt % to about 20 wt %, from about 5 wt % to about 10 wt %, from about 10 wt % to about 50 wt %, from about 10 wt % to about 40 wt %, from about 10 wt % to about 30 wt %, from about 10 wt % to about 20 wt %, from about 20 wt % to about 50 wt %, from about 20 wt % to about 40 wt %, from about 20 wt % to about 30 wt %, from about 30 wt % to about 50 wt %, from about 30 wt % to about 40 wt %, or from about 40 wt % to about 50 wt %.

In some aspects, each of the one or more PEGMA residues may independently have a molecular weight from about 100 to about 8000, for example, from about 100 to about 4000. In some aspects, each of the one or more PEGMA residues have a molecular weight of from about 100 to about 500. In some aspects, each of the one or more PEGMA residues have a molecular weight of from about 100 to about 500, from about 150 to about 500, from about 200 to about 500, from about 250 to about 500, from about 280 to about 500, from about 300 to about 500, from about 380 to about 500, from about 400 to about 500, from about 450 to about 500, from about 100 to about 450, from about 150 to about 450, from about 200 to about 450, from about 250 to about 450, from about 280 to about 450, from about 300 to about 450, from about 380 to about 450, from about 400 to about 450, from about 100 to about 400, from about 150 to about 400, from about 200 to about 400, from about 250 to about 400, from about 280 to about 400, from about 300 to about 400, from about 380 to about 400, from about 100 to about 380, from about 150 to about 380, from about 200 to about 380, from about 250 to about 380, from about 280 to about 380, from about 300 to about 380, from about 100 to about 300, from about 150 to about 300, from about 200 to about 300, from about 250 to about 300, from about 280 to about 300, from about 100 to about 280, from about 150 to about 280, from about 200 to about 280, from about 250 to about 280, from about 100 to about 250, from about 150 to about 250, from about 200 to about 250, from about 100 to 200, from about 150 to 200, or from about 100 to 150.

In various aspects, the disclosed hydrogel can be a copolymer comprising PEGDA and PEGMA residues. A disclosed hydrogel including a polymer comprising PEGDA and PEGMA residues can be formed using the described methods in which polymerization is carried out using PEGDA and PEGMA monomers each at a concentration of greater than or equal to about 1 wt % and less than or equal to about 50 wt %, for example, each from about 1 wt % to about 40 wt %, from about 1 wt % to about 30 wt %, from about 1 wt % to about 20 wt %, from about 1 wt % to about 10 wt %, from about 1 wt % to about 5 wt %, from about 5 wt % to about 50 wt %, from about 5 wt % to about 40 wt %, from about 5 wt % to about 30 wt %, from about 5 wt % to about 20 wt %, from about 5 wt % to about 10 wt %, from about 10 wt % to about 50 wt %, from about 10 wt % to about 40 wt %, from about 10 wt % to about 30 wt %, from about 10 wt % to about 20 wt %, from about 20 wt % to about 50 wt %, from about 20 wt % to about 40 wt %, from about 20 wt % to about 30 wt %, from about 30 wt % to about 50 wt %, from about 30 wt % to about 40 wt %, or from about 40 wt % to about 50 wt %.

In various aspects, a disclosed hydrogel can include a polymer formed from one or more 2-hydroxyethylmethacrylate (HEMA) residues and one or more acrylamide residues; one or more HEMA residues and one or more poly(ethylene glycol)methacrylate (PEGMA) residues; one or more HEMA residues and one or more methacrylic acid residues; one or more HEMA residues and one or more poly(vinyl alcohol) (PVA) residues; or one or more PVA and one or more acrylamide residues. In some aspects, the disclosed hydrogel can be further formed from a disulfide cross-linker such as bisacryloylcystamine.

In order to improve biocompatibility, gels can be dialyzed against deionized water. After dialysis, the formulation can be injected or freeze-dried for storage at room temperature in dry form. Freeze-dried polymers can be rehydrated in aqueous solutions, including balanced salt solutions at physiological, including, but not limited to a pH of about 7.4. In various aspects, an aqueous solution used for rehydration can comprise a pharmaceutically acceptable buffer.

In various aspects, the hydrogels disclosed herein can gel, either in the presence or absence of a disclosed second agent, over a period of from about 15 minutes to about 72 hours. In a further aspect, the gelling time can be from about 30 minutes to about 24 hours.

The disclosed compositions can include a first hydrogel, in which the first hydrogel comprises HEMA, PEGDA, and/or PEGMA residues as disclosed herein, a second hydrogel, and one or more disclosed second agents. The second hydrogel can be any suitable hydrogel as known to the skilled artisan, including, but not limited to, a hydrogel disclosed in U.S. Pat. Appl. Nos. 20050208102, 20050074497, 20090252781, 20140296158, 20130123195, 20150250891, 20160331738, 20160331738, 20170112888, 20180280688, 20180045978, and 20180200340; and in U.S. Pat. Nos. 5,522,888, 5,716,633, 7,939,579, 9,125,807, 9,205,181, 9,775,906, 9,987,367, and 10251954. In some instances, the first hydrogel concentration is essentially about 0 wt %. In other instances, the second hydrogel concentration is essentially about 0 wt %. Representative examples of the second hydrogel as may be used in the disclosed compositions include, but are not limited to, hyaluronic acid, collagen, gelatin, gellan, silk, fibrin, alginate, chitosan, polyacrylamide and methacrylate derivatives thereof, polyacrylic acid and methacrylate derivatives thereof, polyvinyl alcohol, polyethylene glycol and derivatives thereof, polypropylene glycol and derivatives thereof, polymerized ascorbic acid, polycaprolactone, polylactic acid, polyglycolic acid, poly(lactic-co-glycolic acid), or combinations thereof. In some aspects, the compositions may include one or more thermogelling agents, such as, for example, poloxamers.

In some aspects, the polymer composition may include one or more vinyl alcohol residues, one or more acrylamide residues, one or more polyethylene glycol or polyethylene glycol derivative residues, one or more acrylate residues, one or more methacrylate residues, or combinations thereof.

In some aspects, the polymer composition may include one or more residues selected from acrylamide, N-ornithine acrylamide, N-(2-hydroxypropyl)acrylamide, hydroxyethylacrylate, hydroxyethylmethacrylate, polyethylene acrylates, polyethylene methacrylates, N-vinylpyrrolidinone, N-phenylacrylamide, dimethylaminopropyl methacrylamide, acrylic acid, benzylmethacrylamide, methylthioethylacrylamide, caprolactone, lactic acid, glycolic acid, or combinations thereof.

In some aspects, the polymer composition may include hyaluronic acid, collagen, gelatin, gellan, silk, fibrin, alginate, chitosan, polyacrylamides or methacrylate derivatives thereof, polyacrylic acid or methacrylate derivatives thereof, polyvinyl alcohol, polyethylene glycol or derivatives thereof, polypropylene glycol or derivatives thereof, polycaprolactone, polylactic acid, polyglycolic acid, poly(lactic-co-glycolic acid), or combinations thereof.

In some aspects, the polymer composition may include a hyaluronic acid-polyalkylene glycol copolymer, for example, a hyaluronic acid-polyethylene glycol copolymer.

In some aspects, the polymer composition may include one or more residues selected from poly(ethylene glycol)diacrylate (PEDGA), poly(ethylene glycol)methacrylate (PEGMA), 2-hydroxyethylmethacrylate (HEMA), or combinations thereof.

In some aspects, the hydrogel may be in the form of a microgel.

In some particular aspects, the subject may be a human. In other aspects, the disclosed compositions and methods can also be suitable for use in non-human mammals, for example, non-human primates or bovine, equine, porcine, canine, or feline species.

Any suitable joint affected by a joint condition as described herein may be treated by the methods and compositions described herein, including, but not limited to, a knee, a shoulder, a sacroiliac joint, a coxofemoral joint, an ankle, an elbow, an intervertebral disc, an interphalangeal joint, or a wrist. In some aspects, the affected joint to be treated may be selected from an acetabulofemoral joint, an acromioclavicular joint, a femoropatellar joint, a femorotibial joint, a glenohumeral joint, a humeroradial joint, a humeroulnar joint, an interphalangeal joint, a metacarpal joint, a radioulnar joint and a talocrural joint.

Any suitable joint condition may be treated using the methods and compositions described herein. Representative examples of such joint conditions include, but are not limited to, osteoarthritis, rheumatoid arthritis, psoriatic arthritis, gout, tendinitis, bursitis, degenerative disc disease, or Carpal Tunnel syndrome. In some aspects, the joint condition may be selected from: arthroses, chondromalacia patella, isolated chondral defect, juvenile idiopathic arthritis, ligamentous deficiency arthroses, osteoarthritis, degenerative arthritis, degenerative joint disease, osteonecrosis, osteochondritis dissecans, patellar instability, post-ligamentous injury arthritis, post-meniscectomy arthritis, post-meniscectomy arthroses, post-traumatic arthritis, rheumatoid arthritis, and septic arthritis. In some aspects, the joint condition may be an arthropathy selected from: infectious arthritis (such as septic arthritis, tuberculosis arthritis, reactive arthritis); noninfectious arthritis (such as seronegative sponyloarthropathy such as psoriatic arthritis and ankylosing spondylitis, rheumatoid arthritis such as Felty's syndrome, juvenile idiopathic arthritis, adult-onset Still's disease, crystal arthropathy such as gout and chondrocalcinosis, and osteoarthritis); hemarthrosis; synovitis; reactive arthropathy; enteropathic arthropathy; crystal arthropathy (such as gout, pseudogout, chondrocalcinosis, or calcium pyrophosphate deposition disease); diabetic arthropathy; neuropathic arthropathy; and spondylarthropathy.

In another general aspect, injectable compositions are also provided.

In some implementations, the injectable composition may include a hydrogel having one or more residues selected from poly(ethylene glycol)diacrylate (PEDGA), poly(ethylene glycol)methacrylate (PEGMA), 2-hydroxyethylmethacrylate (HEMA), or combinations thereof as described herein; and a reactive oxygen species (ROS) scavenger including a plurality of particles as described herein. In some aspects, the hydrogel may include a PEGDA or PEGMA polymer, as described herein. In some aspects, the hydrogel may include a PEGMA:PEGDA copolymer, as described herein. In some aspects, the hydrogel may include a PEGMA:PEGDA:HEMA copolymer, as described herein. In some aspects, the plurality of particles may include one or more antioxidants, as described herein. In some aspects, the plurality of particles may include one or more therapeutic agents.

In other implementations, the injectable compositions include a hydrogel which may include a mixture or copolymer of a polyalkylene glycol and a second component selected from hyaluronate, fibrin, alginate, or collagen; and a second agent dispersed within the hydrogel selected from one or more antioxidants, one or more reactive oxygen species (ROS) scavengers, or combinations thereof as described herein. In some aspects, the polyalkylene glycol may include a polyethylene glycol. In some aspects, the hydrogel may include a polyethylene glycol-hyaluronate copolymer. In some aspects, the hydrogel may include a polyethylene glycol-fibrin copolymer. In some aspects, the hydrogel may include a polyethylene glycol-alginate copolymer. In some aspects, the hydrogel may include a polyethylene glycol-collagen copolymer. In some aspects, the hydrogel may include hyaluronic acid, collagen, gelatin, gellan, silk, fibrin, alginate, chitosan, polyacrylamides or methacrylate derivatives thereof, polyacrylic acid or methacrylate derivatives thereof, polyvinyl alcohol, polyethylene glycol, polycaprolactone, polylactic acid, polyglycolic acid, poly(lactic-co-glycolic acid), or derivatives thereof, polypropylene glycol or derivatives thereof, or combinations thereof.

The compositions described herein can be provided in any manner which would be deemed suitable for use by injection. In some aspects, the composition can be provided pre-loaded in a syringe prior to injection. In some aspects, the composition can be formulated as a powder that is reconstituted in an aqueous carrier prior to injection. In some aspects, the composition is injected as a liquid and forms a gel at the site of injection. In some aspects, the composition is shear-thinning and injectable as a pre-formed gel.

The present disclosure also pertains to kits comprising one of: (a) the compositions as described herein; (b) the compositions as described herein in a sterile package; or (c) a pre-filled syringe or needle comprising the compositions as described herein; and instructions for administering the compositions as described herein to treat a clinical condition or pathology.

In a further aspect, the disclosed kits can be packaged in a daily dosing regimen (e.g., packaged on cards, packaged with dosing cards, packaged on blisters or blow-molded plastics, etc.). Such packaging promotes products and increases ease of use for administration by a healthcare profession. Such packaging can also reduce potential medical errors. The present disclosure also features such kits further containing instructions for use.

In a further aspect, the present disclosure also provides a pharmaceutical pack or kit comprising one or more packages comprising the disclosed compositions. Associated with such packages can be a notice in the form prescribed by a governmental agency regulating the manufacture, use, or sale of pharmaceuticals or biological products, which notice reflects approval by the agency of manufacture, use, or sale for human administration.

In various aspects, the disclosed kits can also comprise further therapeutic agents, compounds, and/or products co-packaged, co-formulated, and/or co-delivered with other components. For example, a drug manufacturer, a drug reseller, a physician, a compounding shop, or a pharmacist can provide a kit comprising a disclosed composition and another component for delivery to a patient.

It is contemplated that the disclosed kits can be used in connection with the disclosed methods of making, the disclosed methods of using or treating, and/or the disclosed compositions.

In view of the described compositions and methods, hereinbelow are described certain more particular aspects of the disclosure. These particularly recited aspects should not, however, be interpreted to have any limiting effect on any different claims containing different or more general teachings described herein, or that the “particular” aspects are somehow limited in some way other than the inherent meanings of the language and formulas literally used therein.

Aspect 1. A method of treating a joint condition in a subject in need thereof, the method comprising
injecting into an affected joint of the subject a therapeutically effective amount of a therapeutic composition comprising:
a hydrogel; and
a second agent dispersed within the hydrogel selected from one or more antioxidants, one or more reactive oxygen species (ROS) scavengers, or combinations thereof.

Aspect 2. The method of aspect 1, wherein the second agent has a concentration greater than about 1% by weight within the therapeutic composition.

Aspect 3. The method of aspect 1 or aspect 2, wherein the second agent has a concentration from about 1% to about 50% by weight within the therapeutic composition.

Aspect 4. The method of any one of aspects 1-3, wherein the joint condition is selected from osteoarthritis, rheumatoid arthritis, psoriatic arthritis, gout, tendinitis, bursitis, degenerative disc disease, and Carpal Tunnel syndrome.

Aspect 5. The method of any one of aspects 1-4, wherein the one or more antioxidants comprise: N-acetylcysteine; a glutathione; N-selenous acid; sodium selenite; L-carnitine; 0-carotene; vitamin E; ascorbic acid or a derivative thereof; lutein; zeaxanthin; an omega-3 fatty acid; alpha lipoic acid; riboflavin; taurine; uric acid; tyrosine; transferrin; selenium; zinc; superoxide dismutase; glutathione peroxidase; catalase; pigment epithelium-derived factor (PEDF); or combinations thereof.

Aspect 6. The method of any one of aspects 1-5, wherein the one or more antioxidants comprise ascorbic acid or a derivative thereof.

Aspect 7. The method of any one of aspects 1-6, wherein the one or more antioxidants comprise a glutathione.

Aspect 8. The method of any one of aspects 1-5, wherein the one or more reactive oxygen species (ROS) scavengers comprise a plurality of particles.

Aspect 9. The method of aspect 8, wherein the plurality of particles comprise polydopamine (PDA) nanoparticles, cerium oxide particles, polyphenylene sulfide (PPS) particles, phenolic nanoparticles, or combinations thereof.

Aspect 10. The method of aspect 8 or aspect 9, wherein the plurality of particles further comprises one or more antioxidants.

Aspect 11. The method of any one of aspects 8-10, wherein the plurality of particles further comprises one or more therapeutic agents.

Aspect 12. The method of aspect 11, wherein the one or more therapeutic agents are selected from an anti-interleukin antibody, a tyrosine kinase inhibitor, a complement inhibitor, a MAPK inhibitor, a JAK inhibitor, an interferon, an anti-VEGF agent, an anti-NGF agent, a senolytic, or combinations thereof.

Aspect 13. The method of aspect 12, wherein the anti-VEGF agent is selected from aflibercept, axitinib, bevacizumab, cabozantinib, lapatinib, lenvatinib, pazopanib, ponatinib, ramucirumab, ranibizumab, regorafenib, sorafenib, sunitinib, tivozanib, vandetanib, or combinations thereof.

Aspect 14. The method of aspect 12, wherein the one or more therapeutic agents are selected from a nonsteroidal anti-inflammatory drug, a steroid, a disease-modifying antirheumatic drug, or combinations thereof.

Aspect 15. The method of aspect 14, wherein the nonsteroidal anti-inflammatory drug is selected from aspirin, diflunisal, salsalate, ibuprofen, flurobiprofen, dexibuprofen, oxaprozin, naproxen, loxoprofen, fenoprofen, pelubiprofen, ketoprofen, zaltoprofen, dexketoprofen, indomethacin, etodolac, aceclofenac, tolmetin, ketorolac, bromfenac, sulindac, diclofenac, nabumetone, piroxicam, droxicam, phenylbutazone, meloxicam, lornoxicam, tenoxicam, isoxicam, mefenamic acid, flufenamic acid, meclofenamic acid, tolfenamic acid, celocoxib, parecoxib, firocoxib, rofecoxib, lumiracoxib, valdecoxib, etoricoxib, nimesulide, clonixin, and licofelone.

Aspect 16. The method of aspect 14, wherein the steroid is selected from beclomethasone, betamethasone, budesonide, cortisone, dexamethasone, hydrocortisone, methylprednisolone, prednisolone, prednisone, and triamcinolone.

Aspect 17. The method of aspect 14, wherein the disease-modifying antirheumatic drug is selected from abatacept, adalimumab, anakinra, apremilast, azathioprine, baricitinib, certolizumab pegol, chloroquine, ciclosporin, D-penicillamine, etanercept, filgotinib, golimumab, sodium aurothiomalate, auranofin, hydroxychloroquine, infliximab, leflunomide, methotrexate, minocycline, rituximab, sarilumab, secukinumab, sulfasalazine, tocilizumab, tofacitinib, and ustekinumab.

Aspect 18. The method of any one of aspects 1-17, wherein the hydrogel comprises a polymer composition.

Aspect 19. The method of aspect 18, wherein the polymer composition comprises one or more vinyl alcohol residues, one or more acrylamide residues, one or more polyethylene glycol or polyethylene glycol derivative residues, one or more acrylate residues, one or more methacrylate residues, or combinations thereof.

Aspect 20. The method of aspect 18, wherein the polymer composition comprises one or more residues selected from acrylamide, N-ornithine acrylamide, N-(2-hydroxypropyl)acrylamide, hydroxyethylacrylate, hydroxyethylmethacrylate, polyethylene acrylates, polyethylene methacrylates, N-vinylpyrrolidinone, N-phenylacrylamide, dimethylaminopropyl methacrylamide, acrylic acid, benzylmethacrylamide, methylthioethylacrylamide, caprolactone, lactic acid, glycolic acid, or combinations thereof.

Aspect 21. The method of aspect 18, wherein the polymer composition comprises hyaluronic acid, collagen, gelatin, gellan, silk, fibrin, alginate, chitosan, polyacrylamides or methacrylate derivatives thereof, polyacrylic acid or methacrylate derivatives thereof, polyvinyl alcohol, polyethylene glycol or derivatives thereof, polypropylene glycol or derivatives thereof, polycaprolactone, polylactic acid, polyglycolic acid, poly(lactic-co-glycolic acid), or combinations thereof.

Aspect 22. The method of aspect 18, wherein the polymer composition comprises a hyaluronic acid-polyalkylene glycol copolymer.

Aspect 23. The method of aspect 18, wherein the polymer composition comprises one or more residues selected from poly(ethylene glycol)diacrylate (PEDGA), poly(ethylene glycol)methacrylate (PEGMA), 2-hydroxyethylmethacrylate (HEMA), or combinations thereof.

Aspect 24. The method of any one of aspects 1-23, wherein the hydrogel is a microgel.

Aspect 25. The method of any one of aspects 1-24, wherein the therapeutic composition is provided pre-loaded in a syringe prior to injection.

Aspect 26. The method of any one of aspects 1-25, wherein the therapeutic composition is formulated as a powder that is reconstituted in an aqueous carrier prior to injection.

Aspect 27. The method of any one of aspects 1-26, wherein the subject is a human.

Aspect 28. The method of any one of aspects 1-26, wherein the subject is a non-human primate, bovine, equine, porcine, canine, or feline.

Aspect 29. The method of any one of aspects 1-28, wherein the affected joint comprises a knee, a shoulder, a sacroiliac joint, a coxofemoral joint, an ankle, an elbow, an intervertebral disc, an interphalangeal joint, or a wrist.

Aspect 30. An injectable composition comprising:
a hydrogel comprising one or more residues selected from poly(ethylene glycol)diacrylate (PEDGA), poly(ethylene glycol)methacrylate (PEGMA), 2-hydroxyethylmethacrylate (HEMA), or combinations thereof, and
a reactive oxygen species (ROS) scavenger comprising a plurality of particles.

Aspect 31. The injectable composition of aspect 30, wherein the hydrogel comprises a PEGDA or PEGMA polymer.

Aspect 32. The injectable composition of aspect 30, wherein the hydrogel comprises a PEGMA:PEGDA copolymer.

Aspect 33. The injectable composition of aspect 30, wherein the hydrogel comprises a PEGMA:PEGDA:HEMA copolymer.

Aspect 34. The injectable composition of any one of aspects 30-33, wherein the plurality of particles comprises polydopamine (PDA) nanoparticles, cerium oxide nanoparticles, polyphenylene sulfide (PPS) nanoparticles, or combinations thereof.

Aspect 35. The injectable composition of any one of aspects 30-34, wherein the plurality of particles further comprises one or more antioxidants.

Aspect 36. The injectable composition of any one of aspects 30-35, wherein the plurality of particles further comprises one or more therapeutic agents.

Aspect 37. The injectable composition of aspect 36, wherein the one or more therapeutic agents are selected from an anti-interleukin antibody, a tyrosine kinase inhibitor, a complement inhibitor, a MAPK inhibitor, a JAK inhibitor, an interferon, an anti-VEGF agent, an anti-NGF agent, a senolytic, or combinations thereof.

Aspect 38. The injectable composition of aspect 37, wherein the anti-VEGF agent is selected from aflibercept, axitinib, bevacizumab, cabozantinib, lapatinib, lenvatinib, pazopanib, ponatinib, ramucirumab, ranibizumab, regorafenib, sorafenib, sunitinib, tivozanib, vandetanib, or combinations thereof.

Aspect 39. The injectable composition of aspect 37, wherein the one or more therapeutic agents are selected from a nonsteroidal anti-inflammatory drug, a steroid, a disease-modifying antirheumatic drug, or combinations thereof.

Aspect 40. The injectable composition of aspect 39, wherein the nonsteroidal anti-inflammatory drug is selected from aspirin, diflunisal, salsalate, ibuprofen, flurobiprofen, dexibuprofen, oxaprozin, naproxen, loxoprofen, fenoprofen, pelubiprofen, ketoprofen, zaltoprofen, dexketoprofen, indomethacin, etodolac, aceclofenac, tolmetin, ketorolac, bromfenac, sulindac, diclofenac, nabumetone, piroxicam, droxicam, phenylbutazone, meloxicam, lornoxicam, tenoxicam, isoxicam, mefenamic acid, flufenamic acid, meclofenamic acid, tolfenamic acid, celocoxib, parecoxib, firocoxib, rofecoxib, lumiracoxib, valdecoxib, etoricoxib, nimesulide, clonixin, and licofelone.

Aspect 41. The injectable composition of aspect 39, wherein the steroid is selected from beclomethasone, betamethasone, budesonide, cortisone, dexamethasone, hydrocortisone, methylprednisolone, prednisolone, prednisone, and triamcinolone.

Aspect 42. The injectable composition of aspect 39, wherein the disease-modifying antirheumatic drug is selected from abatacept, adalimumab, anakinra, apremilast, azathioprine, baricitinib, certolizumab pegol, chloroquine, ciclosporin, D-penicillamine, etanercept, filgotinib, golimumab, sodium aurothiomalate, auranofin, hydroxychloroquine, infliximab, leflunomide, methotrexate, minocycline, rituximab, sarilumab, secukinumab, sulfasalazine, tocilizumab, tofacitinib, and ustekinumab.

Aspect 43. An injectable composition comprising:
a hydrogel comprising a mixture or copolymer of a polyalkylene glycol and a second component selected from hyaluronate, fibrin, alginate, or collagen; and
a second agent dispersed within the hydrogel selected from one or more antioxidants, one or more reactive oxygen species (ROS) scavengers, or combinations thereof.

Aspect 44. The injectable composition of aspect 43, wherein the polyalkylene glycol comprises a polyethylene glycol.

Aspect 45. The injectable composition of aspect 43 or aspect 44, wherein the hydrogel comprises a polyethylene glycol-hyaluronate copolymer or mixture.

Aspect 46. The injectable composition of aspect 43 or aspect 44, wherein the hydrogel comprises a polyethylene glycol-fibrin copolymer or mixture.

Aspect 47. The injectable composition of aspect 43 or aspect 44, wherein the hydrogel comprises a polyethylene glycol-alginate copolymer or mixture.

Aspect 48. The injectable composition of aspect 43 or aspect 44, wherein the hydrogel comprises a polyethylene glycol-collagen copolymer or mixture.

Aspect 49. The injectable composition of any one of aspects 43-48, wherein the hydrogel further comprises hyaluronic acid, collagen, gelatin, gellan, silk, fibrin, alginate, chitosan, polyacrylamides or methacrylate derivatives thereof, polyacrylic acid or methacrylate derivatives thereof, polyvinyl alcohol, polyethylene glycol or derivatives thereof, polypropylene glycol or derivatives thereof, polylactic acid, polyglycolic acid, poly(lactic-co-glycolic acid), or combinations thereof.

Aspect 50. The injectable composition of any one of aspects 43-49, wherein the one or more antioxidants comprise: N-acetylcysteine; a glutathione; N-selenous acid; sodium selenite; L-carnitine; β-carotene; vitamin E; ascorbic acid or a derivative thereof, lutein; zeaxanthin; an omega-3 fatty acid; alpha lipoic acid; riboflavin; taurine; uric acid; tyrosine; transferrin; selenium; zinc; superoxide dismutase; glutathione peroxidase; catalase; pigment epithelium-derived factor (PEDF); or combinations thereof.

Aspect 51. The injectable composition of any one of aspects 43-50, wherein the one or more antioxidants comprise ascorbic acid or a derivative thereof.

Aspect 52. The injectable composition of any one of aspects 43-51, wherein the one or more antioxidants comprise a glutathione.

Aspect 53. The injectable composition of any one of aspects 43-52, wherein the one or more reactive oxygen species (ROS) scavengers comprise a plurality of particles.

Aspect 54. The injectable composition of aspect 53, wherein the plurality of particles comprise polydopamine (PDA) nanoparticles, cerium oxide particles, polyphenylene sulfide (PPS) particles, or combinations thereof.

Aspect 55. The injectable composition of aspect 53 or aspect 54, wherein the plurality of particles further comprises one or more antioxidants.

Aspect 56. The injectable composition of any one of aspects 53-55, wherein the plurality of particles further comprises one or more therapeutic agents.

Aspect 57. The injectable composition of aspect 56, wherein the one or more therapeutic agents are selected from an anti-interleukin antibody, a tyrosine kinase inhibitor, a complement inhibitor, a MAPK inhibitor, a JAK inhibitor, an interferon, an anti-VEGF agent, an anti-NGF agent, a senolytic, or combinations thereof.

Aspect 58. The injectable composition of aspect 57, wherein the anti-VEGF agent is selected from aflibercept, axitinib, bevacizumab, cabozantinib, lapatinib, lenvatinib, pazopanib, ponatinib, ramucirumab, ranibizumab, regorafenib, sorafenib, sunitinib, tivozanib, vandetanib, or combinations thereof.

Aspect 59. The injectable composition of aspect 56, wherein the one or more therapeutic agents are selected from a nonsteroidal anti-inflammatory drug, a steroid, a disease-modifying antirheumatic drug, or combinations thereof.

Aspect 60. The injectable composition of aspect 59, wherein the nonsteroidal anti-inflammatory drug is selected from aspirin, diflunisal, salsalate, ibuprofen, flurobiprofen, dexibuprofen, oxaprozin, naproxen, loxoprofen, fenoprofen, pelubiprofen, ketoprofen, zaltoprofen, dexketoprofen, indomethacin, etodolac, aceclofenac, tolmetin, ketorolac, bromfenac, sulindac, diclofenac, nabumetone, piroxicam, droxicam, phenylbutazone, meloxicam, lornoxicam, tenoxicam, isoxicam, mefenamic acid, flufenamic acid, meclofenamic acid, tolfenamic acid, celocoxib, parecoxib, firocoxib, rofecoxib, lumiracoxib, valdecoxib, etoricoxib, nimesulide, clonixin, and licofelone.

Aspect 61. The injectable composition of aspect 59, wherein the steroid is selected from beclomethasone, betamethasone, budesonide, cortisone, dexamethasone, hydrocortisone, methylprednisolone, prednisolone, prednisone, and triamcinolone.

Aspect 62. The injectable composition of aspect 59, wherein the disease-modifying antirheumatic drug is selected from abatacept, adalimumab, anakinra, apremilast, azathioprine, baricitinib, certolizumab pegol, chloroquine, ciclosporin, D-penicillamine, etanercept, filgotinib, golimumab, sodium aurothiomalate, auranofin, hydroxychloroquine, infliximab, leflunomide, methotrexate, minocycline, rituximab, sarilumab, secukinumab, sulfasalazine, tocilizumab, tofacitinib, and ustekinumab.

Aspect 63. A method of treating a joint condition in a subject in need thereof, the method comprising injecting into an affected joint of the subject a therapeutically effective amount of a therapeutic composition comprising: one or more reactive oxygen species (ROS) scavengers and one or more therapeutic agents.

Aspect 64. The method of aspect 63, wherein the one or more ROS scavengers comprise a plurality of particles.

Aspect 65. The method of aspect 64, wherein the plurality of particles comprise polydopamine (PDA) nanoparticles, cerium oxide particles, polyphenylene sulfide (PPS) particles, phenolic nanoparticles, or combinations thereof.

Aspect 66. The method of aspect 64 or aspect 65, wherein the plurality of particles further comprises one or more antioxidants.

Aspect 67. The method of any one of aspects 64-66, wherein the plurality of particles encapsulate or incorporate the one or more therapeutic agents.

Aspect 68. The method of any one of aspects 63-67, wherein the one or more therapeutic agents are selected from an anti-interleukin antibody, a tyrosine kinase inhibitor, a complement inhibitor, a MAPK inhibitor, a JAK inhibitor, an interferon, an anti-VEGF agent, an anti-NGF agent, a senolytic, or combinations thereof.

Aspect 69. The method of aspect 68, wherein the anti-VEGF agent is selected from aflibercept, axitinib, bevacizumab, cabozantinib, lapatinib, lenvatinib, pazopanib, ponatinib, ramucirumab, ranibizumab, regorafenib, sorafenib, sunitinib, tivozanib, vandetanib, or combinations thereof.

Aspect 70. The method of any one of aspects 63-67, wherein the one or more therapeutic agents are selected from a nonsteroidal anti-inflammatory drug, a steroid, a disease-modifying antirheumatic drug, or combinations thereof.

Aspect 71. The method of aspect 70, wherein the nonsteroidal anti-inflammatory drug is selected from aspirin, diflunisal, salsalate, ibuprofen, flurobiprofen, dexibuprofen, oxaprozin, naproxen, loxoprofen, fenoprofen, pelubiprofen, ketoprofen, zaltoprofen, dexketoprofen, indomethacin, etodolac, aceclofenac, tolmetin, ketorolac, bromfenac, sulindac, diclofenac, nabumetone, piroxicam, droxicam, phenylbutazone, meloxicam, lornoxicam, tenoxicam, isoxicam, mefenamic acid, flufenamic acid, meclofenamic acid, tolfenamic acid, celocoxib, parecoxib, firocoxib, rofecoxib, lumiracoxib, valdecoxib, etoricoxib, nimesulide, clonixin, and licofelone.

Aspect 72. The method of aspect 70, wherein the steroid is selected from beclomethasone, betamethasone, budesonide, cortisone, dexamethasone, hydrocortisone, methylprednisolone, prednisolone, prednisone, and triamcinolone.

Aspect 73. The method of aspect 70, wherein the disease-modifying antirheumatic drug is selected from abatacept, adalimumab, anakinra, apremilast, azathioprine, baricitinib, certolizumab pegol, chloroquine, ciclosporin, D-penicillamine, etanercept, filgotinib, golimumab, sodium aurothiomalate, auranofin, hydroxychloroquine, infliximab, leflunomide, methotrexate, minocycline, rituximab, sarilumab, secukinumab, sulfasalazine, tocilizumab, tofacitinib, and ustekinumab.

Aspect 74. The method of any one of aspects 63-73, wherein the subject is a human. Aspect 75. The method of any one of aspects 63-73, wherein the subject is a non-human primate, bovine, equine, porcine, canine, or feline.

Aspect 76. The method of any one of aspects 63-75, wherein the affected joint comprises a knee, a shoulder, a sacroiliac joint, a coxofemoral joint, an ankle, an elbow, an intervertebral disc, an interphalangeal joint, or a wrist.

A number of aspects of the disclosure have been described. Nevertheless, it will be understood that various modifications may be made without departing from the spirit and scope of the disclosure. Accordingly, other aspects are within the scope of the following claims.

By way of non-limiting illustration, examples of certain aspects of the present disclosure are given below.

### EXAMPLES

The following examples are put forth to provide those of ordinary skill in the art with a complete disclosure and description of how compositions and methods claimed herein are made and evaluated and are intended to be purely exemplary of the disclosure and are not intended to limit the scope of what the inventors regard as their disclosure. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Example 1. Preparation of Representative Disclosed Hydrogels

Multiple copolymers of HEMA, PEGDA, and PEGMA were synthesized in deionized water (pH 7.4) and screened based on transparency and mechanical properties (Table 1). The hydrogels were formed by free radical polymerization as previously described with modifications. Briefly, HEMA, PEGMA, and PEGDA monomers were dissolved in deionized water and extensively purged with nitrogen gas to remove oxygen molecules that might terminate the reaction prematurely. APS aqueous solution (10% w/v) and TEMED were added as free radical initiator and accelerator at 1:200 and 1:800 v/v, respectively. The solutions were allowed to polymerize for 12 hours. The hydrogels were purified against deionized water for 7 days in dialysis tubing (12-14 kDa MWCO) to remove unreacted monomers and low molecular weight polymer chains. Two optimized formulations were created, namely PEGDA and PEGDA-co-PEGMA hydrogels (Table 1).

TABLE 1
Example 6 - Hydrogel formulations.

| Formulation | PEGDA | PEGDA | PEGMA | HEMA | | | |
|---|---|---|---|---|---|---|---|
| Name | MW | wt % | wt % | wt % | Transparent? | Gel? | Consistency |
| Formulation 1 | 575 | 3% | 3% | 0% | Yes | Yes | Hard |
| Formulation 2 | 575 | 1.2% | 4.8% | 0% | Yes | Yes | Hard |
| Formulation 3 | 575 | 0.3% | 5.7% | 0% | Yes | Yes | Soft |
| Formulation 4 | 575 | 1.2% | 2.4% | 2.4% | No | Yes | Hard |
| Formulation 5 | 575 | 1.2% | 2.1% | 0.3% | Yes | Yes | Hard |
| Formulation 6 | 575 | 1.2% | 0.3% | 2.1% | No | Yes | Hard |
| Formulation 7 | 250 | 1% | 0% | 0% | No | Yes | Hard |
| Formulation 8 | 250 | 0.75% | 0% | 0% | No | No | Liquid |
| | 575 | 0.75% | | | | | |
| Formulation 9 | 250 | 0.75% | 0.75% | 0% | No | No | Liquid |
| Formulation 10 | 575 | 1% | 0% | 1% | No | Yes | Hard |
| Formulation 11 | 250 | 1% | 1% | 1% | No | No | Liquid |
| Formulation 12 | 700 | 1% | 1% | 1% | No | No | Liquid |
| Formulation 13 | 575 | 3% | 0% | 0% | Yes | Yes | Hard |
| Formulation 14 | 575 | 1% | 0% | 0% | Yes | No | Liquid |
| Formulation 15 | 250 | 1.5% | 0% | 0% | No | Yes | Hard |
| Formulation 16 | 575 | 1.5% | 0% | 0% | Yes | No | Liquid |
| Formulation 17 | 700 | 1.5% | 0% | 0% | Yes | No | Liquid |
| Formulation 18 | N/A | 0% | 6% | 0% | Yes | Yes | Soft |
| Formulation 19 | 575 | 2% | 0% | 0% | Yes | Yes | Soft |
| (PEGDA) | | | | | | | |
| Formulation 20 | 575 | 1.5% | 1.5% | 0% | Yes | Yes | Soft |
| (PEGDA-co- | | | | | | | |
| PEGMA) | | | | | | | |

Rheology Determination. Prior to measurement, all hydrogel samples were immersed in DPBS for 7 days to reach equilibrium swelling. The samples were syringed onto the quartz testing stage of a Kinexus ultra+ rheometer (Malvern Instruments Ltd, Worcestershire, UK). A 20-mm parallel plate geometry was lowered onto the hydrogel sample to a working gap of 1 mm, which was determined to provide good contact between the geometry and the hydrogel without damaging the sample (zero normal force). The testing stage was set to 37° C. and a humidifying chamber filled with DPBS was attached around the geometry and testing stage to simulate in vivo conditions and prevent sample dehydration. Amplitude sweep tests were conducted at a frequency of 0.1 Hz and amplitudes ranging from 0.1 to 1000%. Frequency sweep tests with strain amplitude of 1% (found to be within the linear viscoelastic region) were conducted with frequency ranges from 0.01 to 1 Hz to determine the storage modulus (G′) and loss modulus (G″) of the hydrogels. Shear viscosity was evaluated by increasing the shear rate from 0.01 to 1000 s⁻¹. Alternating oscillatory step strains were applied to the hydrogels at a fixed frequency of 0.1 Hz and strains of 10%, 700%, and 1000% with 100 s for each strain interval (H. Wang, et al., Adv. Sci. 2018, 5, 1800711).

Hydrogel Characterization. The equilibrium water content of each hydrogel formulation was determined by drying known amounts of water-swollen hydrogels in a 60° C. oven until no change in weight was detected. The refractive indices of the hydrogels were determined using a refractometer (Sper Scientific, Scottsdale, AZ). The transmittance of the hydrogel was measured using a Varian Cary 50 UV-Visible Spectrophotometer (Agilent Technologies, Santa Clara, CA, USA) at wavelengths ranging from 230 to 900 nm. DPBS was used as a blank. Fourier-transform infrared spectra (FTIR) of the PEGDA and PEGDA-co-PEGMA hydrogels were collected using a Thermo Nicolet Nexus 870 FTIR spectrometer (Thermo Fisher Scientific, Waltham, MA, USA).

Hydrogel Stability. The hydrogels were incubated with DPBS, lysozyme (10,000 U mL⁻¹), or trypsin (0.25%) at 37° C. for up to 4 weeks (S. Santhanam, et al., Acta Biomater. 2016, 43, 327-337). DPBS, lysozyme, or trypsin (1 mL each) was added to PEGDA or PEGDA-co-PEGMA hydrogels (0.5 g). At predetermined times (0, 1, 4, 7, 14, 21, and 28 days), the hydrogels were lyophilized and weighed. The weight stability of the hydrogel samples was determined by the given formula:

$$Weight Stability = \frac{{W}_{t}}{{W}_{0}} * 100$$

where W₀ is the initial weight of the wet hydrogel at time 0 and Wₜ is the weight of the gel at time t (days).

Vitamin C Loading, Stability, and Release. Hydrogels were placed in low molecular weight cut-off dialysis tubing (MWCO 6-8 kDa) and immersed in vitamin C solution (2.2 mM, prepared fresh and changed daily) for 72 hours. A vitamin C concentration of 2.2 mM was chosen as the loading concentration to account for the rapid degradation of vitamin C. To determine the stability of vitamin C in hydrogels, the vitamin C loaded hydrogels were kept at 37° C. At predetermined times (0 and 30 minutes, 1, 2, 4, 8, and 12 hours, 1, 2, 3, 4, and 7 days), the vitamin C remaining in the hydrogel was determined using a Synergy HT multi-mode microplate reader (BioTek, Winooski, VT) at wavelength 265 nm, compared against standard solutions with known concentrations with blank hydrogels as the background reading. To determine vitamin C release, the hydrogels were loaded with vitamin C solution (1% w/v) as aforementioned. A concentration of 1% w/v, or 5.7 mM, was chosen for the release study because lower loading concentrations resulted in lower concentrations of released vitamin C that were too low to be reliably detected. The vitamin C loaded hydrogels (4 mL for each sample) were placed in dialysis tubing (MWCO 6-8 kDa) and submerged in DPBS (100 mL). At predetermined times as described above, DPBS solution (1 mL) was withdrawn to determine the concentration of vitamin C released, and fresh DPBS (1 mL) was added to maintain sink condition.

Cell Viability and ROS Activity Assays. ARPE-19 and LEC were seeded in 96-well plates at 1×10⁴ cells per well in DMEM/F-12 and DMEM, respectively, supplemented with 10% FCS and 1% Pen Strep for 24 h at 37° C. in 5% CO₂ humidified atmosphere. The hydrogels were submerged in 70% ethanol for 1 hour to sterilize, rinsed with deionized water 3 times for 1 hour each to remove the residual ethanol, and mixed well with serum-free and phenol red-free DMEM at a hydrogel concentration of 10% w/v (J. Chang, et al., J Mater Chem B. 2015, 3, 1097-1105; Y. Tao, et al., Acta Biomater. 2013, 9, 5022-5030; M. Annaka, et al., Biomacromolecules. 2011, 12, 4011-4021; and S. Lamponi, et al., J Biomater Sci Polym Ed. 2012, 23, 555-575). The culture medium in each well was removed and medium (100 μL) with/without hydrogel and with/without vitamin C (2.2 mM) was added to each well and incubated for 24 hours. Hydrogen peroxide (10 μL, 200 μM final concentration) was added to half of the wells, and DPBS (10 μL) was added to the remaining wells as a control (A. Heckelen, et al., Acta Ophthalmol Scand. 2004, 82, 564-568; and H. S. Lee, et al., Invest. Ophthalmol. Vis. Sci. 2017, 58, 1196-1207). The well plates were incubated for 30 minutes. CellTiter-Glo luminescent cell viability assay was conducted according to the manufacturer's protocol. Briefly, the well plates were equilibrated to room temperature for 30 minutes. CellTiter-Glo Reagent (100 μL) was added to each well, and the contents were mixed for 10 minutes using an orbital shaker. The well plates were incubated at room temperature for 10 minutes before the luminescent signal was measured using the Synergy HT multi-mode microplate reader. ROS activity was detected using DCF. Briefly, DCF (100 μL, 20 μM final concentration) was added to each well, and the contents were incubated at room temperature for 30 minutes (Y. Ou, et al., Chem Biol Interact. 2009, 179, 103-109). The fluorescence signal was measured with excitation and emission wavelengths of 485 and 525 nm, respectively, using a TECAN M200 Plate Reader (Mannedorf, Switzerland).

Statistical Analysis. Data are expressed as mean±standard error (SE). Statistical analyses were implemented with Minitab software (version 18.1; Minitab, Inc., State College, PA). One-way ANOVA, with post-hoc pairwise comparison using Tukey test, was used to analyze the rheological data, the hydrogel stability data, and the cell viability and ROS activity of the ARPE-19 and LEC. The null hypotheses stated that there is no difference between the groups for each test. An alpha value of 0.05 was used for statistical significance.

### Example 2. Preparation of Drug-Loaded PDA Nanoparticles

Dopamine hydrochloride and fluorescein isothiocyanate conjugate (FITC)-bovine serum album (BSA) were obtained from Sigma-Aldrich (St. Louis, MO). Bevacizumab (Avastin) was purchased from Genentech, Inc. (San Francisco, CA). Colorimetric (3-(4,5-dimethylthiazol-2-yl)-5-(3-carboxymethoxyphenyl)-2-(4-sulfophenyl)-2H-tetrazolium) (MTS) assay, 2′,7′-dichlorodihydrofluorescein diacetate (DCFH-DA), and lipopolysaccharide (LPS) were purchased from Fisher Scientific Inc. (Hampton, NH). Rhodamine phalloidin, 4′,6-diamidino-2-phenylindole (DAPI), and Amplex red hydrogen peroxide/peroxidase assay kit were purchased from Thermo Fisher Scientific Inc. (Columbus, OH). Human retinal pigment epithelial cells (ARPE-19 cells, CRL2302) were purchased from American Type Culture Collection (Rockville, MD). Human umbilical vein endothelial cells (HUVECs, C0035C), Geltrex LDEV-free reduced growth factor basement membrane matrix, and Invitrogen human VEGF ELISA kit were purchased from Thermo Fisher Scientific Inc. (Waltham, MA). All other reagents used in this study were analytical grade.

### Synthesis of Drug-Loaded PDA Nanoparticles

PDA nanoparticles and drug-loaded PDA nanoparticles were prepared based on methods reported previously with some modifications. Briefly, 100 mg dopamine hydrochloride was added to 50 mL deionized (DI) water containing 420 μL 1N sodium hydroxide solution, which was stirred at 500 rpm for 3-hour polymerization. FITC-BSA was used as a model drug for the particle characterization and the release study of PDA nanoparticles, and bevacizumab, the commonly used anti-VEGF medication for treating wet AMD, was loaded afterward. 100 mg FITC-BSA or bevacizumab was then added to the PDA solution, which was stirred for another 21 hours. Later, the prepared nanoparticles were collected by centrifuge and washed five times with DI water until all the proteins on the surface of the nanoparticles were removed. Then, the PDA nanoparticles were lyophilized and stored at 4° C. until further use.

### Characterization

The morphological characterization of PDA nanoparticles was done by transmission electron microscopy (TEM) (FEI Tecnai G2 Biotwin), and the diameter was determined by measuring at least 100 nanoparticles from TEM images using Image J (NIH). The Zetasizer (Malvern Nano ZS) was also used to determine the hydrodynamic diameter of PDA nanoparticles and drug-loaded nanoparticles. The absorption of PDA nanoparticles at different concentrations was determined by UV-Vis spectrophotometer (Agilent Cary 100).

### Degradation

The degradation of PDA nanoparticles was determined by mixing 200 μg/mL PDA nanoparticles with 10 mM hydrogen peroxide (H₂O₂) diluted in phosphate-buffered saline (PBS) at 37° C. At specific time points, the absorption of degraded PDA nanoparticles was assessed by a UV-Vis spectrophotometer, and their morphological properties were examined by TEM.

### Drug Release and Loading of PDA Nanoparticles

For the oxidative response release, the 1 mg/mL FITC-BSA loaded PDA nanoparticles were dispersed in PBS with 10 mM, 5 mM, 2 mM, 1 mM, and 0 mM (control group) H₂O₂ and incubated at 37° C. At specific time points, the eluent was collected, and 1 mL of fresh PBS containing different amounts of H₂O₂ was added to the PDA nanoparticles. The eluted FITC-BSA in the supernatant was assessed by a multi-mode microplate reader (BioTek Synergy HTX) (Eₑₓ=485 nm, Eₑₘ=525 nm). Similarly, the release of bevacizumab triggered by different concentrations of H₂O₂ was also studied. The active bevacizumab released from PDA nanoparticles triggered by 10 mM and 0 mM H₂O₂ was assessed by enzyme-linked immunosorbent assay (ELISA). These concentrations were selected to evaluate maximum and minimum release. All the experiments were done in triplicate. The drug loading rate of PDA nanoparticles was determined by dissolving the FITC-BSA or bevacizumab loaded nanoparticles in H₂O₂ and its loading rate was calculated as the total amount of FITC-BSA or bevacizumab loaded into the particles/the mass weight of PDA nanoparticles*100%.

### Cytotoxicity

Cell viability in the presence of PDA nanoparticles was conducted with human retinal pigmented epithelial (ARPE-19) cells. To be specific, the ARPE-19 cells were seeded in 48-well plates at a density of 4×10⁴ cells/well. The cells were incubated with 0 μg/mL, 1 μg/mL, 2 μg/mL, 5 μg/mL, 10 μg/mL, 20 μg/mL, 50 μg/mL, 100 μg/mL, 200 μg/mL, 500 μg/mL, 1000 μg/mL of PDA nanoparticles for another 24 hours. Then, the particles were removed and cells were treated by MTS reagent for 3 hours at 37° C. The absorbance measurement at 490 nm was obtained by using the microplate reader and normalized to the blank group. The cells were additionally stained by live/dead cell assay and observed by fluorescent microscopy (Nikon, Eclipse TS100) to further determine the influence of PDA nanoparticles on cell viability. All experiments were repeated three times, and the result was displayed as average±standard deviation.

### Cellular Uptake of PDA Nanoparticles

Considering the cell viability of PDA nanoparticles, cellular uptake was determined by treating the ARPE-19 cells with 10 g/mL PDA nanoparticles. To better visualize the nanoparticles, the PDA nanoparticles were loaded with FITC-BSA. After 24-hour incubation, the cells were stained by 4′,6-diamidino-2-phenylindole (DAPI) and rhodamine-phalloidin and observed under a confocal fluorescence microscope (Zeiss Axio Observer Z1).

### ROS Scavenging by PDA Nanoparticles

To investigate the capability of nanoparticles in scavenging ROS, Amplex red assay was utilized to assess the consumption rate of H₂O₂ by PDA nanoparticles. Briefly, 1 mg blank PDA nanoparticles were treated with 10 mM H₂O₂ and incubated at 37° C. At 30 min, 1 h, 2 h, 4 h, 8 h, 1 day, 2 days, 4 days, and 1 week, the 1 μL of the solution was retrieved and diluted in PBS which was then quantified by using the Amplex red kit. To further confirm the intracellular ROS scavenging capability of PDA nanoparticles, H₂O₂ and LPS (lipopolysaccharide) were used to introduce inflammation using the retinal pigment cell line. Briefly, ARPE-19 cells were seeded in 24-well plates and 96-well plates at a density of 2×10⁵ cells/mL 24 hours before the study. Then, 200 M H₂O₂ or 50 g/mL LPS with 0 g/mL, 1 g/mL, 2 g/mL, 5 g/mL, 10 g/mL, 20 g/mL, or 50 g/mL PDA nanoparticles were added to the cell and incubated for 24 hours. Afterward, the cells were thoroughly washed with fresh media and treated with 10 μM DCFH-DA for 1 hour. Cells were visualized by a fluorescent microscope, and the intracellular ROS levels revealed by the fluorescent intensity were assessed by a microplate reader and normalized to that of the control group (100%). Each experiment was done in triplicate. The result was displayed as an average±standard deviation.

### VEGF Inhibition Effect of PDA Nanoparticles

To assess the effect of PDA nanoparticles on reducing angiogenesis, a human VEGF ELISA kit was used to quantify the amount of VEGF released from the cells induced by H₂O₂.⁴² Briefly, ARPE-19 cells were seeded in 24-well plates for 24 hours. Then, the cells were incubated with blank media (control group), 200 mM H₂O₂, 200 mM H₂O₂ and 10 μg/mL PDA nanoparticles, 200 mM H₂O₂ and 10 μg/mL bevacizumab loaded nanoparticles, as well as 200 mM H₂O₂ and 5 μg/mL free bevacizumab. After 24 hours, the active VEGF in the media was determined by ELISA. The tube formation assay is a rapid method to assess the angiogenesis of HUVECs induced by ROS. Under oxidative stress, ARPE-19 cells were co-cultured with HUVECs to investigate the influence of H₂O₂ and drug-loaded PDA nanoparticles on the development of the three-dimensional capillary structure. Briefly, ARPE-19 cells at a concentration of 2×10⁵ cells/mL were seeded on the 12 trans-well plate insert 24 hours before the experiment. Afterward, intracellular inflammation was induced by 200 μM H₂O₂. 10 μg/mL PDA nanoparticles, 10 μg/mL bevacizumab loaded nanoparticles, and 5 μg/mL bevacizumab were also added to the cells to inhibit the production of VEGF, respectively. After 12 hours, the well plate was coated with the basement membrane matrix allowing for the growth of HUVECs. Then, 800 μL of HUVECs stained by Calcein AM were added to each well and incubated for another 12 hours. The tube formation induced by VEGF secreted from ARPE-19 cells and H₂O₂ was observed by a fluorescent microscope and the tube length was analyzed using Image J. The tube lengths of experimental groups were normalized to the control group (100%). Each condition was done in triplicate. The result was shown as the average±standard deviation.

### Statistical Analysis

Statistical analysis was performed by one-way ANOVA with post-hoc Tukey test by OriginLab. The significance was p<0.05.

The following references are hereby by incorporated in their entireties:
1. Pengfei Jiang et al. “Controlled release of anti-VEGF by redox-responsive polydopamine nanoparticles” Nanoscale, 2020, 12:17298.
2. Marakand V. Risbud et al. “Role of Cytokines in Intervertebral Disc Degeneration: Pain and Disc-content” Nat Rev Rheumatol, 2014, 10(1):44-56.
3. Tetsuro Ohba et al. “TNF-α-Induced NF-κB Signaling Reverses Age-Related Declines in VEGF Induction and Angiogenic Activity in Intervertebral Disc Tissues” Journal of Orthopaedic Research, 2009, 229.
4. Jun Sato et al. “Inhibiting Vascular Endothelial Growth Factor in Injured Intervertebral Discs Attenuates Pain-Related Neuropeptide Expression in Dorsal Root Ganglia in Rats” Asian Spine J, 2017, 11(4); 556-561.
5. WO2020/163872
6. WO2021/262645

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method of treating a joint condition in a subject in need thereof, the method comprising
injecting into an affected joint of the subject a therapeutically effective amount of a therapeutic composition comprising:
a hydrogel; and
a second agent dispersed within the hydrogel selected from one or more antioxidants, one or more reactive oxygen species (ROS) scavengers, or combinations thereof.

2. (canceled)

3. The method of claim 1, wherein the second agent has a concentration from about 1% to about 50% by weight within the therapeutic composition.

4. The method of claim 1, wherein the joint condition is selected from osteoarthritis, rheumatoid arthritis, psoriatic arthritis, gout, tendinitis, bursitis, degenerative disc disease, and Carpal Tunnel syndrome.

5. The method of claim 1, wherein the one or more antioxidants comprise: N-acetylcysteine; a glutathione; N-selenous acid; sodium selenite; L-carnitine; β-carotene; vitamin E; ascorbic acid or a derivative thereof, lutein; zeaxanthin; an omega-3 fatty acid; alpha lipoic acid; riboflavin; taurine; uric acid; tyrosine; transferrin; selenium; zinc; superoxide dismutase; glutathione peroxidase; catalase; pigment epithelium-derived factor (PEDF); or combinations thereof.

6. The method of claim 1, wherein the one or more antioxidants comprise ascorbic acid or a derivative thereof, and/or
wherein the one or more antioxidants comprise a glutathione.

7. (canceled)

8. The method of claim 1, wherein the one or more reactive oxygen species (ROS) scavengers comprise a plurality of particles.

9. The method of claim 8, wherein the plurality of particles comprise polydopamine (PDA) nanoparticles, cerium oxide particles, polyphenylene sulfide (PPS) particles, phenolic nanoparticles, or combinations thereof.

10. The method of claim 8, wherein the plurality of particles further comprises one or more antioxidants, and/or
wherein the plurality of particles further comprises one or more therapeutic agents.

11. (canceled)

12. The method of claim 10, wherein the one or more therapeutic agents are selected from an anti-interleukin antibody, a tyrosine kinase inhibitor, a complement inhibitor, a MAPK inhibitor, a JAK inhibitor, an interferon, an anti-VEGF agent, an anti-NGF agent, a senolytic, or combinations thereof.

13. The method of claim 12, wherein the anti-VEGF agent is selected from aflibercept, axitinib, bevacizumab, cabozantinib, lapatinib, lenvatinib, pazopanib, ponatinib, ramucirumab, ranibizumab, regorafenib, sorafenib, sunitinib, tivozanib, vandetanib, or combinations thereof.

14. The method of claim 12, wherein the one or more therapeutic agents are selected from a nonsteroidal anti-inflammatory drug, a steroid, a disease-modifying antirheumatic drug, or combinations thereof.

15. The method of claim 14, wherein the nonsteroidal anti-inflammatory drug is selected from aspirin, diflunisal, salsalate, ibuprofen, flurobiprofen, dexibuprofen, oxaprozin, naproxen, loxoprofen, fenoprofen, pelubiprofen, ketoprofen, zaltoprofen, dexketoprofen, indomethacin, etodolac, aceclofenac, tolmetin, ketorolac, bromfenac, sulindac, diclofenac, nabumetone, piroxicam, droxicam, phenylbutazone, meloxicam, lornoxicam, tenoxicam, isoxicam, mefenamic acid, flufenamic acid, meclofenamic acid, tolfenamic acid, celocoxib, parecoxib, firocoxib, rofecoxib, lumiracoxib, valdecoxib, etoricoxib, nimesulide, clonixin, and licofelone, or
wherein the steroid is selected from beclomethasone, betamethasone, budesonide, cortisone, dexamethasone, hydrocortisone, methylprednisolone, prednisolone, prednisone, and triamcinolone, or
wherein the disease-modifying antirheumatic drug is selected from abatacept, adalimumab, anakinra, apremilast, azathioprine, baricitinib, certolizumab pegol, chloroquine, ciclosporin, D-penicillamine, etanercept, filgotinib, golimumab, sodium aurothiomalate, auranofin, hydroxychloroquine, infliximab, leflunomide, methotrexate, minocycline, rituximab, sarilumab, secukinumab, sulfasalazine, tocilizumab, tofacitinib, and ustekinumab.

16-17. (canceled)

18. The method of claim 1, wherein the hydrogel comprises a polymer composition.

19. The method of claim 18, wherein the polymer composition comprises one or more vinyl alcohol residues, one or more acrylamide residues, one or more polyethylene glycol or polyethylene glycol derivative residues, one or more acrylate residues, one or more methacrylate residues, or combinations thereof, or
wherein the polymer composition comprises one or more residues selected from acrylamide, N-ornithine acrylamide, N-(2-hydroxypropyl)acrylamide, hydroxyethylacrylate, hydroxyethylmethacrylate, polyethylene acrylates, polyethylene methacrylates, N-vinylpyrrolidinone, N-phenylacrylamide, dimethylaminopropyl methacrylamide, acrylic acid, benzylmethacrylamide, methylthioethylacrylamide, caprolactone, lactic acid, glycolic acid, or combinations thereof, or
wherein the polymer composition comprises hyaluronic acid, collagen, gelatin, gellan, silk, fibrin, alginate, chitosan, polyacrylamides or methacrylate derivatives thereof, polyacrylic acid or methacrylate derivatives thereof, polyvinyl alcohol, polyethylene glycol or derivatives thereof, polypropylene glycol or derivatives thereof, polycaprolactone, polylactic acid, polyglycolic acid, poly(lactic-co-glycolic acid), or combinations thereof, or
wherein the polymer composition comprises a hyaluronic acid-polyalkylene glycol copolymer, or
wherein the polymer composition comprises one or more residues selected from poly(ethylene glycol)diacrylate (PEDGA), poly(ethylene glycol)methacrylate (PEGMA), 2-hydroxyethylmethacrylate (HEMA), or combinations thereof.

20-23. (canceled)

24. The method of claim 1, wherein the hydrogel is a microgel.

25. The method of claim 1, wherein the therapeutic composition is provided pre-loaded in a syringe prior to injection, and/or
wherein the therapeutic composition is formulated as a powder that is reconstituted in an aqueous carrier prior to injection.

26. (canceled)

27. The method of claim 1, wherein the subject is a human, or
wherein the subject is a non-human primate, bovine, equine, porcine, canine, or feline.

28. (canceled)

29. The method of claim 1, wherein the affected joint comprises a knee, a shoulder, a sacroiliac joint, a coxofemoral joint, an ankle, an elbow, an intervertebral disc, an interphalangeal joint, or a wrist.

30. An injectable composition comprising:
a hydrogel comprising one or more residues selected from poly(ethylene glycol)diacrylate (PEDGA), poly(ethylene glycol)methacrylate (PEGMA), 2-hydroxyethylmethacrylate (HEMA), or combinations thereof, and
a reactive oxygen species (ROS) scavenger comprising a plurality of particles.

31-42. (canceled)

43. An injectable composition comprising:
a hydrogel comprising a mixture or copolymer of a polyalkylene glycol and a second component selected from hyaluronate, fibrin, alginate, or collagen; and
a second agent dispersed within the hydrogel selected from one or more antioxidants, one or more reactive oxygen species (ROS) scavengers, or combinations thereof.

44-62. (canceled)

63. A method of treating a joint condition in a subject in need thereof, the method comprising injecting into an affected joint of the subject a therapeutically effective amount of a therapeutic composition comprising: one or more reactive oxygen species (ROS) scavengers and one or more therapeutic agents.

64-76. (canceled)
